(19) United States
(12) Patent Application Publication
(10) Pub. No.: US 2007/0270358 A1
(43) Pub. Date: Nov. 22, 2007

(54) Cosmetic and/or Pharmaceutical Composition for the Treatment of the Rosacea

(76) Inventor: Gianfranco de Paoliambrosi, Salo (Brescia) (IT)

Correspondence Address: MCGLEW & TUTTLE, PC, P.O. BOX 9227, SCARBOROUGH STATION, SCARBOROUGH, NY, 10510-9227, US

(21) Appl. No.: 11/569,317

(22) PCT Filed: May 18, 2005
(86) PCT No.: PCT/IT05/00280

§ 371 Date: Nov. 17, 2006

(30) Foreign Application Priority Data
May 24, 2004 (IT) BS2004A000068

(51) Int. Cl. A61K 31/10; A61K 31/192; A61K 31/352; A61K 31/355; A61K 31/4164; A61K 31/44; A61K 31/56; A61K 31/65; A61K 31/70; A61K 31/7048; A61P 17/06; A61P 17/08; A61P 17/10

(52) U.S. Cl. 514/29; 514/154; 514/171; 514/23; 514/27; 514/291; 514/398; 514/456; 514/458; 514/474; 514/568; 514/711

(57) ABSTRACT

The invention concerns a cosmetic, pharmaceutical, or medical device composition based on dimethyl sulfone to be applied on the skin, both integral and damaged, for the treatment of rosacea, acne, psoriasis, atopic dermatitis, dermatitis seborrheica, erythema intending by erythema also cutaneous flushing as it is called in the cosmetics field. The invention concerns also the use of dimethyl sulfone in combination with substances having anti-bacterial and/or anti-biotic activity, and metronidazole, flavonoids, vitamins, cortisone agents, tacrolimus for said treatment.

### FIELD OF THE INVENTION

The present invention has as object a new composition for cosmetic or pharmaceutical treatment to be applied on the skin, both integral and damaged, or on the mucosa for the treatment of rosacea, acne, psoriasis, atopic dermatitis, dermatitis seborrheica, erythema in general of the skin, and in particular erythema caused by gamma radiation, X rays and ultraviolet radiation, intending by erythema also cutaneous flushing as it is known as in the cosmetics field.

### State of the Art

Cutaneous inflammation is a complex phenomena encompassing the most common dermatosis. Rosacea, acne, atopic dermatitis, dermatitis seborrheica, erythema of the skin are characterised by an inflammatory component.

Just as an indication, here following is described the importance of erythema in the development of a specific pathology: rosacea.

Rosacea is a chronic cutaneous disorder that interests primarily the central face, cheeks, chin, nose and forehead, often characterised by remissions and exacerbations and include a series of symptoms such as lushing, permanent erythema, teleangiectasia, edema, papules, pustules, and ocular lesions.

Rosacea occurs in both men and women both and normally begins after the age of 30.

The nosology of rosacea is not well established. The scientific committee of the “National Rosacea Society” in the United States has developed a classification of four subtypes of Rosacea.

Subtype 1—Erythematotelangiectatic Rosacea

It is mainly characterised by flushing and persistent central facial erythema. The appearance of telangiectases is common but not essential for a diagnosis of this subtype. Central facial edema, stinging and burning sensations, and roughness or scaling may also be reported. A history of flushing alone is common among patients with erythematotelangiectatic rosacea.

Subtype 2—Papulopustular Rosacea

This subtype is characterised by persistent central facial erythema with transient papules or pustules or both but not only localised in a central facial distribution. In many aspects it may be confused with acne vulgaris, except that comedones are absent. Rosacea and acne may occur concomitantly. This subtype of rosacea is often associated with subtype 1.

Subtype 3—Phymatous Rosacea

This subtype of rosacea includes thickening skin, irregular surface nodularities, and enlargement of the nose (rhinophyma), even if phymatous rosacea may occur in other locations such as the cheeks, forehead, chin and ears. This subtype of rosacea is often seen in combination with subtypes 1 and 2, with the presence of persistent erythema and telangiectases.

Subtype 4—Ocular Rosacea

The diagnosis of ocular rosacea should be considered when a patient has one of the following symptoms: interpalpebral hyperemia and conjunctival, burning, stinging, light sensitivity, dryness, telangiectases of the conjunctiva and lid margin and periocular erythema. Ocular rosacea is often diagnosed when the symptoms of rosacea are present.

Early treatment of rosacea is of fundamental importance in impeding its course. When rosacea is not treated, very often it worsens and the possibilities of therapeutic success lessen.

If it is true that their exist different therapeutic means for controlling the inflammatory lesions, such as papules or pustules, nothing exists which is able to control erythema which, as seen previously, most probably represents the start of the rosacea syndrome.

Another aspect to be taken into consideration is the one represented by the toxic action of radiation in determining the intensity of erythema and its passage from intermittent to persistent.

### DESCRIPTION OF THE INVENTION

Previously the same inventor proposed the use of dimethyl sulfone and a composition containing this substance for the treatment of cutaneous irritation provoked by chemical, physical, bacterial and viral agents, generically.

This invention concerns more specifically and explicitly the use of dimethyl sulfone (also known as Sulfonylbismethane; DMSO₂, methylsulfone; methylsulfonylmethane, C₂H₆O₂S; molecular weight 94.13; CH₃SO₂CH₃), opportunely transmitted for topical use, for the treatment of rosacea, acne, psoriasis, atopic dermatitis, dermatitis seborrheica, and erythema of the skin.

In this invention dimethyl sulfone is used to inhibit intermittent and persistent erythema in persons suffering from rosacea belonging to the subtypes 1,2,3,4.

Dimethyl sulfone also carries out a marked cyto-protective action both on keratinocytes and on fibroblasts from the noxious effect exerted by radiation, in particular both A and B type ultraviolet rays.

The invention refers in particular to a composition for the above use that is characterised by the fact that it contains, as an active principle, dimethyl sulfone in a percentage in weight from 0.1% to 90%, if possible between 1% to 50%, preferably between 2% to 10%. The percentage of use of dimethyl sulfone generally depends on the type of application and on the cosmetic and/or pharmaceutical form used.

Within the field of this invention both the compositions containing dimethyl sulfone as the sole active principle, in association with cosmetic and/or pharmaceutical excipients, and the compositions in which dimethyl sulfone is used in combination with anti-bacteria and/or antibiotic agents, such as metronidazole, azelaic acid, benzoyl peroxide, clindamycin, erythromycin, sulphacetamide, doxycycline, minocycline, tetracycline, azithromycin, triethyl citrate including relative salts, esters and amides, both in racemic mixture and in dextrorotatory and levorotatory forms, and in possible cis and trans forms, are included.

Also included in the field of this invention are the compositions containing dimethyl sulfone as the active principle in combination with agents which are part of the flavonoids chemical group (both in the pure state and containing plants, parts of plants or dry extracts, alcohols, hydro alcohols, glycerics, glycolics etc.), such as silymarin, quercitin, hesperidia, diosmin and with vitamins, such as ascorbic acid, vitamin P, tocopherol, pantenol, retinol, retinaldehyde, retinoic acid, salicylic acid, lactic acid, pyruvic acid, mandelic acid, glycocholic acid, citric acid, trichloroacetic acid, dichloroacetic acid, monochloroacetic acid, acetic acid—including relative salts, esters and amides, both in racemic mixture and in dextrorotatory and levorotatory forms, and in possible cis and trans forms.

Always included in the field of this invention are the compositions containing dimethyl sulfone in combination with agents which are part of the cortisone group, such as halcinonide, alclometasone, alfametilprednisolone, beclomethasone, budesonide, clobetasol, clobetasone, dexamethasone, desoximetasone, diflucortolone, flumethasone, fluocinolone, diflucortolone, flucinonide, fluocortin, fluocortolone, hydrocortisone, methylprednisolone, mometasone, triamcinolone, including relative salts, esters and amides, both in racemic mixture and in dextrorotatory and levorotatory forms, and in possible cis and trans forms.

In the field of this invention are the compositions containing dimethyl sulfone in combination with tacrolimus, including relative salts, esters and amides, both in racemic mixture and in dextrorotatory and levorotatory forms, and in possible cis and trans forms.

When dimethyl sulfone is the only active principle of the composition, it is included in variable quantities between 0.1% and 90% in weight, if possible between 1% to 50% in weight, preferably between 2% to 10% in weight.

When dimethyl sulfone is used in combination with anti-bacteria and/or antibiotic agents, the quantity of dimethyl sulfone is between 0.1% and 50% in weight and the quantity of anti-bacteria and/or antibiotic agent is between 0.005% and 30% in weight.

If the antibiotic agent is metronidazole, the dimethyl sulfone is used in a quantity in weight from 0.5% to 80% and the metronidazole in a quantity in weight from 0.05% to 10%, preferably from 0.1% to 2%.

When dimethyl sulfone is used in combination with a flavonoid, the dimethyl sulfone is used in a quantity in weight from 0.1% to 50% and the flavonoid in a quantity in weight from 0.1% to 15%.

When dimethyl sulfone is used in combination with vitamins, the dimethyl sulfone is used in a quantity in weight from 0.1% to 50% and the quantity in weight of vitamins is from 0.001% to 15%.

When dimethyl sulfone is used in combination with cortisones, the dimethyl sulfone is used in a quantity in weight from 0.1% to 50% and the quantity in weight of cortisone agents is from 0.001% to 15%.

When dimethyl sulfone is used in combination with tacrolimus, dimethyl sulfone is used in a quantity in weight from 0.1% to 50% and the quantity in weight of tacrolimus is from 0.001% to 1%.

### EXPERIMENTAL PART

Evaluation in vitro of the antioxidant function if dimethyl sulfone through the study of its antiradical action on cell cultures of human keratinocytes.

“Free radicals” mean a chemical species capable of independent existence, that contains one or more unpaired electrons and, therefore, highly reactive in regards to other molecules. Hydroxy and peroxy radicals are very dangerous and according to a theory put forward by Harman in 1954, cause damage to membranes, alter proteins, inactivate enzymes and produce senile pigment. The cytotoxic action of free radicals is contrasted by cell defensive systems represented by enzymes that behave as ANTIOXIDANTS such as SOD (superoxide dismutase) which fights the action of peroxy radicals converting them unto H₂O₂ (hydrogen peroxide), CATALYSIS and GLUTATHIONE PEROXIDASE that convert H₂O₂ into pure water before other complexes use it as a substratum to generate hydroxy radicals.

The free radicals generate both during normal metabolic reactions that occur in the cell, and by induction from external agents such as pharmaceutical products, foodstuffs, pesticides, tobacco smoke, radiations.

Exposure to light in persons suffering from rosacea, represent an important factor in determining the intensity of the erythema. Numerous experiments have shown that the average life of diploid human cell cultures subjected to stress, toxic substances and UV rays is increased with the addition of antioxidant substances in the culture medium, prompting dermatologists and cosmetologists to protect the skin from the danger of free radicals, by integrating cosmetics used daily for personal care with an antioxidant ingredient. In this regard, Vitamins A, C and E are among the most effective agents that protect the cells from lipid peroxidation. Vitamin C, in particular if administered in high doses, is able to interact very rapidly both with superoxides and hydroxy radicals, is easily procured through extraction from fruit and vegetables, it is not toxic even if taken in very high doses, it activates all our vital processes, protects cell membranes, increases the resistance of the skin against harmful external agents, is present in cell exchanges assisting the absorption of nutritive substances, and delays the cell ageing process.

The evaluation test is carried out to establish if the tested product, in different concentrations, has in vitro, the antioxidant activity sought after. For this reason the capacity of the composition of the invention to neutralize the reactive species of the oxygen (ROS) and to inhibit the death of the cell, is tested. This capacity is exhaustive in determining the control of the formation of erythema in rosacea.

The in vitro test carried out on skin tissue cells has been found to be a test method able to give specific information on the reactions that may occur in vivo.

Keratinocytes are characteristic epidermis cells and play a key role in all the functions of the skin. In these experiments keratinocytes coming from biopsies of healthy voluntary donors were used.

To evaluate the antioxidant role played by the test substance, it was decided to use two types of tests.

A first test enables evaluation of whether the substance being examined possesses neutralization activity in connection with the reactive oxygen species (ROS) by in vitro measuring of the quantity of ROS produced by the cells following induced oxidative stress, compared with non-treated controls.

A second test, through the determination of the vitality of the cells using the MTT method, enables evaluation of the total damage suffered by the cells (in the absence of and following oxidative stress) and the protective effect produced by the substance tested in different concentrations.

### DOSAGE OF ROS

The substance to be tested was diluted in a salt solution to the final concentrations required. The dichlorofluorescein acetate (DCF) was diluted apart in a special buffer. The DCF reacts with the free radicals if present giving rise to a fluorescent derivative, and the fluorometry reading gives a quantity related to the presence of this substance in the cells analyzed. A sufficient number of cells (30,000 cells/well) is sown in the wells of a 90 well plate. After a pre-incubation period overnight with the different sample concentrations, the culture medium is sucked out of the wells and replaced with 500 μl of DCF solution. The plates are placed in a CO₂ thermo regulator at 37° C. to incubate for 15 minutes. At this point the DCF solution is discarded. After a 5 minute period of exposure to UV rays, the oxidative stress is interrupted, and the fluorometry reading taken. The lamp used in the experiments reproduces the solar spectrum with a constant UVA emission field of between 315 and 400 nm. The emission of UVB is appropriately shielded to avoid direct cytotoxic damage to the cell cultures. The plate containing the cells is irradiated at room temperature with an intensity of 1.7 mW/cm² of UVA (5 J/cm²).

Reading of the fluorometry is carried out at the excitation wave length of 485 nm and emission of 530 nm directly on the plates (Toxicol. Letters 1997-93: 47-54).

Measuring Cell Vitality Using MTT

Before and following exposure to UV rays, an MTT test is carried out to evaluate the toxic impact on the cell energetic system (mitochondrions) compared with cells not protected from oxidative stress a cells not exposed to stress. The MTT test is simple, accurate and gives reproducible results. This method, originally developed by Mossman (1993), is based on a yellow substance in solution. The mitochondrial dehydrogenize of live cells is able to cut the tetrazolium ring causing the formation of insoluble purple coloured salts. The crystals can de dissolved in acidified isopropanol and the purple solution formed can be spectrophotometrically dosed. An increase/decrease in number of the live cells can be evaluated as corresponding to the increase or decrease in the optical absorption due to formazano salts, giving a quantification of the global cytotoxic event.

### EVALUATION OF THE ANTI-RADICAL ACTIVITY AND CELLULAR VITALITY

Control negative (C−): cells not treated
Control positive (C+): cells exposed to UV without protective treatment
Samples:
cells exposed to UV in the presence of protective substances:
Vitamin C 0.1 mg/ml (anti-oxidant of comparison)
SOL.—Solution 20% dimethyl sulfone

### PROTECTION FROM ROS

After 5 minutes exposure to UV

The results are given in terms of fluorescence, that are directly proportional to the quantity of ROS

| | | | | MSM SOL. | MSM SOL. | MSM SOL. | MSM SOL. | MSM SOL. | MSM SOL. | MSM SOL. |
|---|---|---|---|---|---|---|---|---|---|---|
| | | | VIT C | 20% 2.5 | 20% 0.83 | 20% 0.28 | 20% 0.09 | 20% 0.03 | 20% 0.01 | 20% 0.003 |
| Sample | C+ | C− | 0.1 mg/ml | mg/ml | mg/ml | mg/ml | mg/ml | mg/ml | mg/ml | mg/ml |
| Average | 29719 | 856 | 20837 | 17782 | 16848 | 18675 | 17117 | 19686 | 18887 | 17820 |
| Value | | | | | | | | | | |
| Standard | 7084 | 21 | 1584 | 662 | 3566 | 1293 | 5548 | 3059 | 6209 | 3765 |
| deviation | | | | | | | | | | |

The anti-radical protection percentage offered by the substance is calculated using the following formula:

| | MSM SOL. | MSM SOL. | MSM SOL | MSM SOL. | MSM SOL. | MSM SOL. | MSM SOL. |
|---|---|---|---|---|---|---|---|
| | 20% 2.5 | 20% 0.83 | 20% 0.28 | 20% 0.09 | 20% 0.03 | 20% 0.01 | 20% 0.003 |
| Sample | mg/ml | mg/ml | mg/ml | mg/ml | mg/ml | mg/ml | mg/ml |
| Average | 41.4 | 44.6 | 38.3 | 43.7 | 34.8 | 37.5 | 41.2 |
| Value | | | | | | | |

Cellular vitality as evaluated in the absence of oxidative stress (UV−) and after 5 minutes of exposure to U(UV+)

Absorption values (O.D) at 540 nm (in proportion to the number of cells). Evaluation of cellular vitality in the absence of oxidative stress

| | | | MSM SOL. | MSM SOL. | MSM SOL. | MSM SOL. | MSM SOL. | MSM SOL. | MSM SOL. |
|---|---|---|---|---|---|---|---|---|---|
| | | VIT C | 20% 2.5 | 20% 0.83 | 20% 0.28 | 20% 0.09 | 20% 0.03 | 20% 0.01 | 20% 0.003 |
| Sample | C− | 0.1 mg/ml | mg/ml | mg/ml | mg/ml | mg/ml | mg/ml | mg/ml | mg/ml. |
| Average | 2893 | 2.977 | 2.785 | 2.735 | 2.858 | 2.816 | 2.926 | 3.052 | 2.961 |
| Value | | | | | | | | | |
| Standard | 0.010 | 0.383 | 0.052 | 0.041 | 0.069 | 0.063 | 0.107 | 0.189 | 0.055 |
| deviation | | | | | | | | | |

Evaluation of cellular vitality after 5 minutes exposure to UV

| | | | MSM SOL. | MSM SOL | MSM SOL. | MSM SOL. | MSM SOL. | MSM SOL. | MSM SOL. |
|---|---|---|---|---|---|---|---|---|---|
| | | VIT C | 20% 2.5 | 20% 0.83 | 20% 0.28 | 20% 0.09 | 20% 0.03 | 20% 0.01 | 20% 0.003 |
| Sample | C+ | 0.1 mg/ml | mg/ml | mg/ml | mg/ml | mg/ml | mg/ml | mg/ml | mg/ml. |
| Average | 2.768 | 2.904 | 2.777 | 2.838 | 2.832 | 2.987 | 2.940 | 2.977 | 3.020 |
| Value | | | | | | | | | |
| Standard | 0.077 | 0.048 | 0.142 | 0.034 | 0.156 | 0.094 | 0.110 | 0.037 | 0.083 |
| deviation | | | | | | | | | |

Summary of the data collected and evaluation of the antioxidant capacity and protection of the product of the invention subjected to experimentation.

From the above it results that dimethyl sulfone, at the concentration of between 0.003 and 2.5 mg/ml, possesses antioxidant activity and significantly reduces the presence of reactive oxygen species (ROS) in Keratinocytes cultures subjected to oxidative stress.

Dimethyl sulfone, a the concentration of between 0.003 and 2.5 mg/ml, possesses cytoprotective activity: in fact following exposure to UV radiation cellular vitality of treated Keratinocytes is greater compared with non-treated Keratinocytes.

### EXAMPLES OF FORMULATIONS

Preparation 1

Pharmaceutical emulsion for topical use based on dimethyl sulfone and metronidazole:

| N^{o} | Description | % p/p a |
|---|---|---|
| | Phase A) | |
| 01 | Steareth 2 | 2.00 |
| 02 | Steareth 21 | 3.00 |
| 03 | Ppg-15 stearyl ether | 10.00 |
| 04 | Stearic acid | 5.00 |
| | BHT | 0.01 |
| | PHASE B) | |
| 05 | Metronidazole | 0.75 |
| 06 | Dimethyl sulfone | 5.00 |
| 07 | Ethyl alcohol | 5.00 |
| 08 | Preservatives | q.s. |
| 09 | WATER | q.s. |

Method of preparation: Heat PHASE A) at +80° C. Heat 09 at +75° C. and mix with PHASE A) to form an emulsion. Mix 05, 06, 07, 08 to prepare PHASE B) to heat at +40° C. and then mix with the emulsion prepared previously.
Preparation 2

Pharmaceutical emulsion for topical use based on dimethyl sulfone and erythromycin

| N^{o} | Description | % p/p a |
|---|---|---|
| | Phase A) | |
| 01 | Steareth 2 | 2.00 |
| 02 | Steareth 21 | 3.00 |
| 03 | Ppg-15 stearyl ether | 10.00 |
| 04 | Stearic acid | 5.00 |
| | BHT | 0.01 |
| | PHASE B) | |
| 05 | Erythromycin | 1.00 |
| 06 | Dimethyl sulfone | 5.00 |
| 07 | Ethyl alcohol | 5.00 |
| 08 | Preservatives | qs |
| 09 | WATER | qs |

Method of preparation: Heat PHASE A) at +80° C. Heat 09 at +75° C. and mix with PHASE A) to form an emulsion. Mix 05, 06, 07, 08 to prepare PHASE B) to heat at +40° C. and then mix with the emulsion prepared previously.
Preparation 3

Pharmaceutical emulsion for topical use based on dimethyl sulfone and gentamicin:

| N^{o} | Description | % p/p a |
|---|---|---|
| | Phase A) | |
| 01 | Steareth 2 | 2.00 |
| 02 | Steareth 21 | 3.00 |
| 03 | Ppg-15 stearyl ether | 10.00 |
| 04 | Stearic acid | 5.00 |
| | BHT | 0.01 |
| | PHASE B) | |
| 05 | Gentamicin sulphate | 0.16 |
| 06 | Dimethyl sulfone | 5.00 |
| 07 | Ethyl alcohol | 5.00 |
| 08 | Preservatives | qs |
| 09 | WATER | qs |

Method of preparation: Heat PHASE A) at +80° C. Heat 09 at +75° C. and mix with PHASE A) to form an emulsion. Mix 05, 06, 07, 08 to prepare PHASE B) to heat at +40° C. and then mix with the emulsion prepared previously.

Preparation 4

Cosmetic and/or Pharmaceutical solution for topical use based on dimethyl sulfone and Triethyl citrate:

| N^{o} | Description | % p/p a |
|---|---|---|
| 01 | Dimethyl sulfone | 20.00 |
| 02 | Triethyl citrate | 5.00 |
| 03 | Propylenic glycol | 3.00 |
| 04 | Glycerol | 2.00 |
| 05 | Preservatives | qs |
| 06 | Water qs | 100 |

Method of preparation: dissolve 01 in 06 previously heated at +50° C. In the solution resulting, mix 02,03,04 with 05.

Preparation 5

Single-phase pharmaceutical solution for topical use based on dimethyl sulfone and clindamycin:

| N^{o} | Description | % p/p a |
|---|---|---|
| 01 | Dimethyl sulfone | 50.00 |
| 02 | Clindamycin HCl | 1.00 |
| 03 | Propylenic glycol | 5.00 |
| 05 | Dimethyl Isosorbide | 20.00 |
| 06 | Water qs | 100 |

Method of preparation: dissolve 01+02 in 06; mix 03+04 to the resulting solution.

Preparation 6

Cosmetic emulsion for topical use based on dimethyl sulfone and silymarin:

| N^{o} | Description | % in weight |
|---|---|---|
| | Phase A) | |
| 01 | Steareth 2 | 2.00 |
| 02 | Steareth 21 | 3.00 |
| 03 | CETEARYL GLUCOSIDE, CETEARYL ALCOHOL | 10.00 |
| 04 | Ppg-15 stearyl ether | 5.00 |
| 05 | Stearic acid | 0.01 |
| 06 | CETEARYL ETHYLEXANOATE | |
| 07 | DIMETHICONE | 0.16 |
| 08 | BHT | 5.00 |
| 09 | TOCOPHERYL ACETATE | 5.00 |
| 10 | ASCORBYL TETRAISOPALMITATE | qs |
| 11 | PETROLATUM | qs |
| 12 | DIMETHICONE COPOLYOL | |
| 13 | STEARYL GLYCYRRHETINATE | |
| 14 | PARAFFINUM LIQUIDUM | |
| | PHASE B) | |
| 15 | SILYBUM MARIANUM (SILYMARIN) | 1.00 |
| 16 | PROPYLENE GLYCOL | 5.00 |
| 17 | DIMETHYL SULFONE | 5.00 |
| 18 | WATER | 15.00 |
| | PHASE C) | |
| 19 | Preservatives | qs |
| 20 | Water qs | 100 |

Method of preparation: Mix the ingredients of PHASE A) and heat at +80° C. Prepare PHASE C) dissolving 19 in 20, heat at 75° C. and then mix with PHASE A). Homogenize for 10 min. then allow to cool while being agitated, down to 45° C. Prepare the phase B) dissolving 15 into 16 (previously heated to 80° C.) and 17 into 18 (previously heated at 70° C.); mix the two solutions of PHASE B) allow them to cool down to a temperature of 50° C., then mix them with the emulsion resulting from the mixture of PHASE B) and PHASE C).
Preparation 7

Pharmaceutical solution for topical use based on dimethyl sulfone and hydrocortisone:

| N^{o} | Description | % in weight |
|---|---|---|
| 01 | Dimethyl sulfone | 10.00 |
| 02 | Dimethyl Isosorbide | 25.00 |
| 03 | Hydrocortisone | 1.5 |
| 05 | Preservatives | qs |
| 06 | Water qs | 100 |

Method of preparation: dissolve 01+03 in 02 then mix with 05 in which 04 has been previously dissolved.

Preparation 8

A cosmetic solution for topical use based on dimethyl sulfone and pantenol:

| N^{o} | Description | % in weight |
|---|---|---|
| 01 | Dimethyl sulfone | 10.00 |
| 02 | Pantenol | 25.00 |
| 03 | Glycol propylene | 1.5 |
| 04 | Preservatives | qs |
| 05 | Water qs | 100 |

Method of preparation: dissolve 01 in which 05 has previously been dissolved, then mix together with 02+03.

1. Use of dimethyl sulfone in the preparation of a pharmaceutical, cosmetic or medical devices for topical use to prevent and/or cure acne rosacea, acne, psoriasis, atopic dermatitis, dermatitis seborrheica, erythema caused both by chemical and physical agents such as gamma radiation, X-rays and ultraviolet rays.

2. Pharmaceutical and/or cosmetic composition for use according to claim 1, including, as an active principle, a pharmaceutically effective quantity of dimethyl sulfone.

3. A composition according to claim 2, in which the dimethyl sulfone is present in a percentage in weight of from 0.1% to 90%

4. A composition according to claim 3, in which the dimethyl sulfone is present in a percentage in weight of from 1% to 50%, preferably between 2% and 10%.

5. A composition according to claim 2, in which the dimethyl sulfone is used in combination with at least one active anti-bacterial and/or antibiotic substance.

6. A composition according to claim 5, in which the dimethyl sulfone is combined with metronidazole, and in which dimethyl sulfone is in a quantity in weight from 0.5% to 80% and the metronidazole is in a quantity in weight from 0.05 to 10%, preferably from 0.1% to 2%.

7. A composition according to claim 5, in which the active anti-bacterial and/or antibiotic substances include azelaic acid, benzoyl peroxide, clindamycin, erythromycin, sulphacetamide, doxycycline, minocycline, tetracycline, azithromycin, triethyl citrate, including relative salts, esters and amides, both in racemic mixture and in dextrorotatory and levorotatory forms, and in possible cis and trans forms.

8. A composition according to claim 7, in which the quantity of dimethyl sulfone is between 0.1% and 50% in weight and the quantity of anti-bacterial and/or antibiotic substance is between 0.005% and 30% in weight.

9. A composition according to claim 2, in which the dimethyl sulfone is used in combination with one or more agents, part of the flavonoid chemical group.

10. A composition according to claim 9, in which the agents of the flavonoid group include silymarin, quercitin, hesperidin, diosmin, relative salts, esters and amides, both in racemic mixture and in dextrorotatory and levorotatory forms, and in possible cis and trans forms.

11. A composition according to claim 9 in which the quantity of dimethyl sulfone is between 0.1% and 50% in weight and the quantity of flavonoid is between 0.1% and 15% in weight.

12. A composition according to claim 2, in which the dimethyl sulfone is used in combination with vitamins.

13. A composition according to claim 12, in which the vitamins include ascorbic acid, vitamin P, tocopherol, pantenol, retinol, retinaldehyde, retinoic acid, salicylic acid, lactic acid, pyruvic acid, mandelic acid, glycocholic acid, citric acid, trichloroacetic acid, dichloroacetic acid, monochloroacetic acid, acetic acid, including relative salts, esters and amides, both in racemic mixture and in dextrorotatory and levorotatory forms, and in possible cis and trans forms.

14. A composition according to claim 12 in which the quantity of dimethyl sulfone is between 0.1% and 50% in weight and the quantity of vitamins is between 0.001% and 15% in weight.

15. A composition according to claim 2, in which the dimethyl sulfone is used in combination with cortisone agents.

16. A composition according to claim 15, in which the cortisone agents include halcinonide, alclometasone, alfametilprednisolone, beclomethasone, budesonide, clobetasol, clobetasone, dexamethasone, desoximetasone, diflucortolone, flumethasone, fluocinolone, diflucortolone, flucinonide, fluocortin, fluocortolone, hydrocortisone, methylprednisolone, mometasone, triamcinolone, including relative salts, esters and amides, both in racemic mixture and in dextrorotatory and levorotatory forms, and in possible cis and trans forms.

17. A composition according to claim 15, in which the quantity of dimethyl sulfone is from 0.1% to 50% in weight and the quantity in weight of the cortisone agents is from 0.001% to 15%.

18. A composition according to claim 2, in which the dimethyl sulfone is used in combination with tacrolimus, including relative salts, esters and amides, both in racemic mixture and in dextrorotatory and levorotatory forms, and in possible cis and trans forms.

19. A composition according to claim 18, in which the quantity in weight of dimethyl sulfone is from 0.1% to 50% and the quantity in weight of tacrolimus is from 0.001% to 1%.
